(19) United States
(12) Patent Application Publication
(10) Pub. No.: US 2007/0196451 A1
(43) Pub. Date: Aug. 23, 2007

(54) Extendable rolled delivery system

(75) Inventors: Anurag Singhal, Natick, MA (US); Robert Herrmann, Boston, MA (US); Eun-Hyun Jang, Allston, MA (US); Young-Ho Song, Natick, MA (US)

Correspondence Address: KENYON & KENYON LLP, 1500 K STREET N.W., SUITE 700, WASHINGTON, DC, 20005, US

(73) Assignee: Boston Scientific Scimed, Inc., Maple Grove, MN (US)

(21) Appl. No.: 11/358,117

(22) Filed: Feb. 22, 2006

(51) Int. Cl. A61M 31/00; A61K 9/70

(52) U.S. Cl. 424/443; 424/448; 604/500

(57) ABSTRACT

The present invention relates to a device and method for delivery of a patch, graft, implant, therapeutic agent, or other device onto tissue using a rolled delivery mechanism. In one embodiment, a rolled delivery mechanism is provided for delivering a patch to tissue when the rolled delivery mechanism is unrolled. In another embodiment, a rolled delivery mechanism is provided for delivering therapeutic agent to a target tissue.

### TECHNICAL FIELD

The present invention relates to the delivery of a patch, graft, implant, therapeutic agent, or other material to a target site of an organic vessel.

### BACKGROUND

The delivery of therapeutic agents to diseased muscle or other tissue is an important, often repeated, procedure in the practice of modem medicine. Therapeutic agents, including therapeutic drugs and genetic material, may be used to treat, regenerate, or otherwise affect the muscle surface or the interior of the muscle itself. Such therapy can promote revascularization and create new formation of muscle, such as the myocardium of the heart. For example, many of the treatments for a failing heart due to congestive heart failure entail the delivery of therapeutic agents, growth factors, nucleic acids, gene transfection agents, or cellular transplants, e.g. fetal cardiomyocytes, allogeneic cardiomyocytes, allogeneic or autologous myocytes, and other potentially pluripotential cells from autologous or allogeneic bone marrow or stem cells.

Current methods for delivering therapeutic agents to muscle, such as the heart muscle, entail injecting directly into the muscle a genetic cell or therapeutic drug. Delivery of therapeutic agents has been proposed or achieved using medical devices such as catheters, needle devices and various coated implantable devices such as stents. The cells and agents can be injected directly or can be formulated into gels, sealants, or microparticles for injection.

Certain areas of the body, such as between an organ and the surrounding membrane, present particular difficulties for effective implantation of a patch, implant or graft, or application of therapeutic agents, due to the restricted space involved. For example, the region between the pericardium and the myocardium of the heart is particularly space-limited and difficult to reach and treat using traditional catheters such as balloon-type catheters. The application of a patch to tissue by a balloon catheter generally requires a catheter with an expanded diameter at least equal to the width of the patch, and a catheter with a length at least equal to the length of the patch. Thus, it is difficult to place a patch of a large size in confined, space-limited locations for treatment with balloon-type catheters, and the overall efficacy of a therapy may be reduced.

Accordingly, there is a need for a system to allow placement of patches, grafts, implants and therapeutic agents in space-limited and sensitive areas. Further, there is a need for a system that allows the insertion and placement of relatively large patches, grafts and implants using small profile medical delivery devices.

### SUMMARY OF THE INVENTION

The present invention relates to a system for the delivery of therapeutic agent in a confined space, wherein the system requires little space for delivery of the therapeutic agent.

In one embodiment of the present invention, a system for delivering therapeutic agent in a confined space is provided, wherein the system comprises a rolled delivery mechanism at the end of a catheter, endoscope, thorascope, or other device. A sheet comprising a patch, therapeutic agent, gel, or other device or substance may be disposed on the rolled portion, for example on one side of the rolled portion, such that the device or substance to be deposited may be rolled in place with the rolled portion of the rolled delivery mechanism, and placed on the surface of a muscle, organ, or other tissue when the rolled portion is unrolled. The rolled portion may be unrolled via fluid pressure, application of heat, mechanical means, or other methods. As the rolled delivery mechanism is unrolled, pressure from the mechanism may cause the therapeutic agent, patch, or graft to be delivered to the desired location.

In an alternative embodiment of the present invention, the rolled delivery mechanism itself may be a patch or graft that is to applied to the desired location, such that the rolled delivery mechanism, patch or graft detaches from the catheter, endoscope, thorascope or other device after the rolled delivery mechanism is unrolled. The detached rolled delivery mechanism remains at the target tissue site acting as the patch or graft after the catheter, thorascope or endoscope is removed.

In alternative embodiments of the present invention, a system for delivering therapeutic agent in a confined space is provided, wherein a rolled delivery mechanism is disposed on the end of a catheter, endoscope, thorascope, or other device, and the rolled delivery mechanism is covered by a sheath. The sheath may facilitate delivery of the rolled delivery mechanism to the targeted tissue site, and may constrain the rolled delivery mechanism. The rolled delivery mechanism may comprise a patch or other therapeutic agent to be delivered to tissue. The rolled delivery mechanism may be disposed within the sheath, such that the longitudinal axis of the rolled portion is perpendicular to the longitudinal axis of the catheter or other device. The rolled delivery mechanism may also be disposed within the sheath such that its longitudinal axis is parallel to the longitudinal axis of the catheter or other device.

In another alternative embodiment, the patch, graft, or other device to be delivered to tissue may be folded when rolled within the rolled delivery mechanism, such that when the delivery mechanism is unrolled the patch may unroll and further unfold in order to be placed in a confined region, thereby permitting a patch having a large width to be delivered to the target site.

In another alternative embodiment, the patch, graft, or other device to be delivered to tissue may comprise a metal or shape-memory material. When the delivery mechanism is unrolled, the patch may assume a pre-defined shape.

In some embodiments of the invention, a system for delivery of a therapeutic agent in a confined space is provided, wherein a rolled delivery mechanism is disposed on the end of a catheter or other device, wherein the delivery mechanism comprises a patch containing a therapeutic agent. The delivery mechanism may be unrolled, for example with fluid pressure, application of heat, mechanical means, or other methods.

In some embodiments of the invention, a delivery mechanism is provided that expands primarily in a single dimension. Other delivery methods, such as balloon-type catheters, require expansion in several dimensions and therefore cannot be used to deliver therapeutic agents to confined areas of the body, or to deliver large patches to tissue without requiring a large-sized catheter or delivery device. The present invention therefore provides a way to deliver a therapeutic agent to confined spaces such as between an organ and the surrounding membrane, and along the outside or inside surface of organs and other structures. It is therefore advantageous, for example, in the treatment of infarction, ulcers, and wounds, and as part of cancer therapies. The patch may also be used to deliver therapeutic agent, allowing therapeutic agents to be administered to the interior and the surface of a muscle or other tissue.

### BRIEF DESCRIPTION OF THE DRAWINGS

FIG. 1 shows an enlarged side view of a delivery system according to one embodiment of the present invention.

FIG. 2 shows an enlarged side view of a delivery system according to another embodiment of the present invention.

FIG. 3 shows an enlarged side view of a delivery system according to an embodiment of the present invention.

FIG. 4 shows an enlarged side view of a delivery system according to an embodiment of the present invention.

FIG. 5 shows an enlarged perspective view of a delivery system according to an embodiment of the present invention.

FIG. 6 shows an embodiment of the present invention positioned near tissue of a patient to be treated.

FIG. 7 shows an embodiment of the present invention positioned near tissue of a patient to be treated.

FIG. 8 shows an embodiment of the present invention positioned near tissue of a patient to be treated.

FIG. 9 shows an embodiment of the present invention positioned near tissue of a patient to be treated.

FIG. 10 shows an enlarged side view of a delivery system showing the rolled portion unrolled.

### DETAILED DESCRIPTION

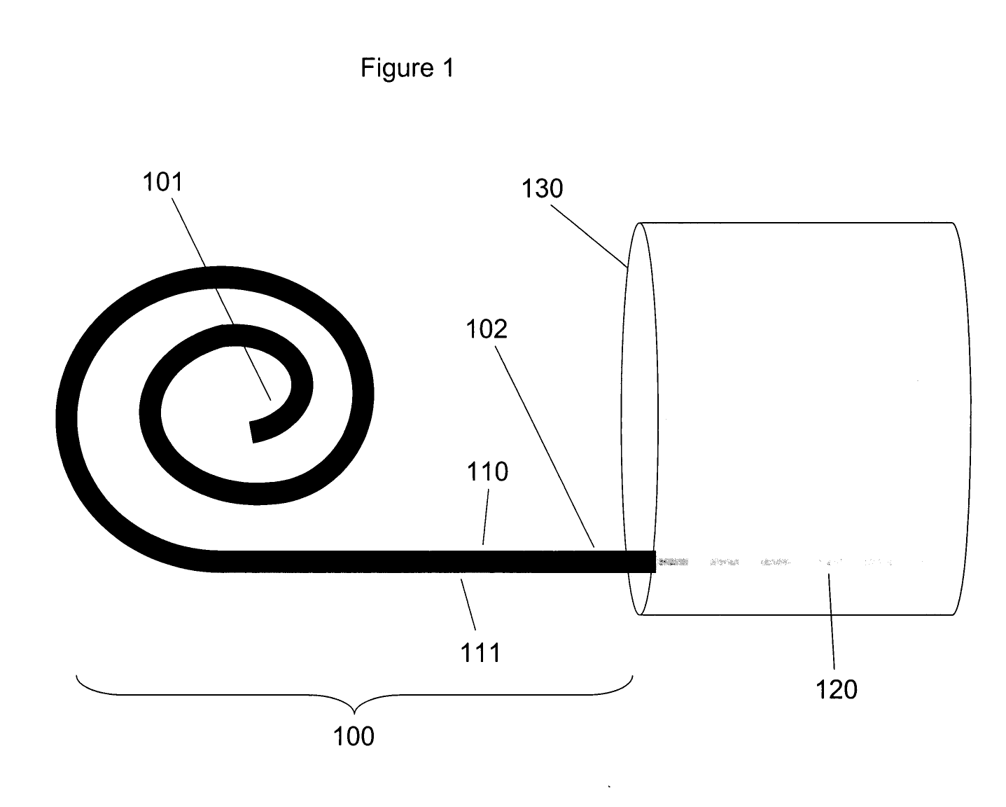

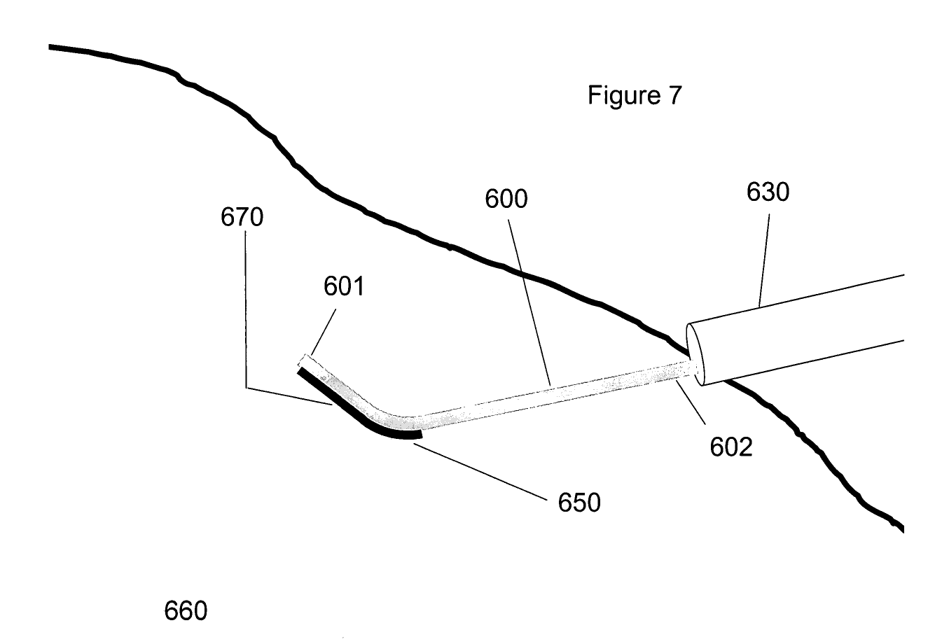

FIG. 1 shows one embodiment of the present invention. Catheter 130 has rolled delivery mechanism 100 disposed at one end. The rolled delivery mechanism is a device for delivering a patch, graft or implant, or a therapeutic agent to a desired location or site within a muscle, organ, tissue or other structure within the body. To deliver the patch or therapeutic agent, the delivery mechanism may be unrolled as depicted in FIG. 7. The delivery mechanism is rolled along its longitudinal axis from a distal end (depicted as distal end 101 in FIG. 1, or distal end 601 in FIG. 7) to a proximal end (depicted as proximal end 102 in FIG. 1, or proximal end 602 in FIG. 7) to facilitate delivery of the patch or therapeutic agent to the desired location. The distal end of the rolled delivery mechanism is farthest from the operator of the device. The proximal end of the rolled delivery device is the end closest to the operator of a device. Delivery mechanism 100 may be extendable from the interior of catheter 130. In other embodiments, delivery mechanism 100 may be attached to the end of catheter 130. In preferred embodiments, delivery mechanism 100 may be attached to catheter 130 and fluidly communicate with catheter 130 such that fluid pressure may be used to unroll delivery mechanism 100. Such fluid may include a gas or a liquid directed through the catheter. For example, a liquid such as saline may be used to unroll delivery mechanism 100, much like the inflation methods for balloon catheters. In other embodiments, a mechanism such as a push rod 120 inserted through catheter 130, may be used to extend delivery mechanism 100. One of ordinary skill in the art would understand that push rod 120 may be positioned within the delivery mechanism 100 (as shown in FIG. 1), or positioned adjacent delivery mechanism 100 (not shown) and extend towards the distal end 101 to unroll delivery mechanism 100.

One of ordinary skill in the art would understand that catheter 130 may be introduced surgically or thorascopically to a treatment site (such as at the epicardial surface of the heart), or may be introduced interventionally to a treatment site (such as at the endocardial surface of the heart). One skilled in the art would appreciate that catheter 130 may be a thorascope or endoscope instead of a balloon catheter for non-interventional surgical procedures.

Referring to FIG. 1, the rolled delivery mechanism 100 has a concave surface, such as surface 110, when in a rolled position. This surface 110 is an “inner surface”. The convex surface of rolled delivery mechanism 100, such as surface 111, is an “outer surface”. A therapeutic agent or a layer containing a therapeutic agent may be disposed on an outer surface such as surface 111 for ease of positioning. Therapeutic agent may be disposed on surface 111, and may comprise a gel, paste, or other substance. When delivery mechanism 100 is unrolled, pressure from delivery mechanism 100 may cause outer surface 111 to contact the intended delivery site, allowing transfer of a therapeutic agent onto the targeted site.

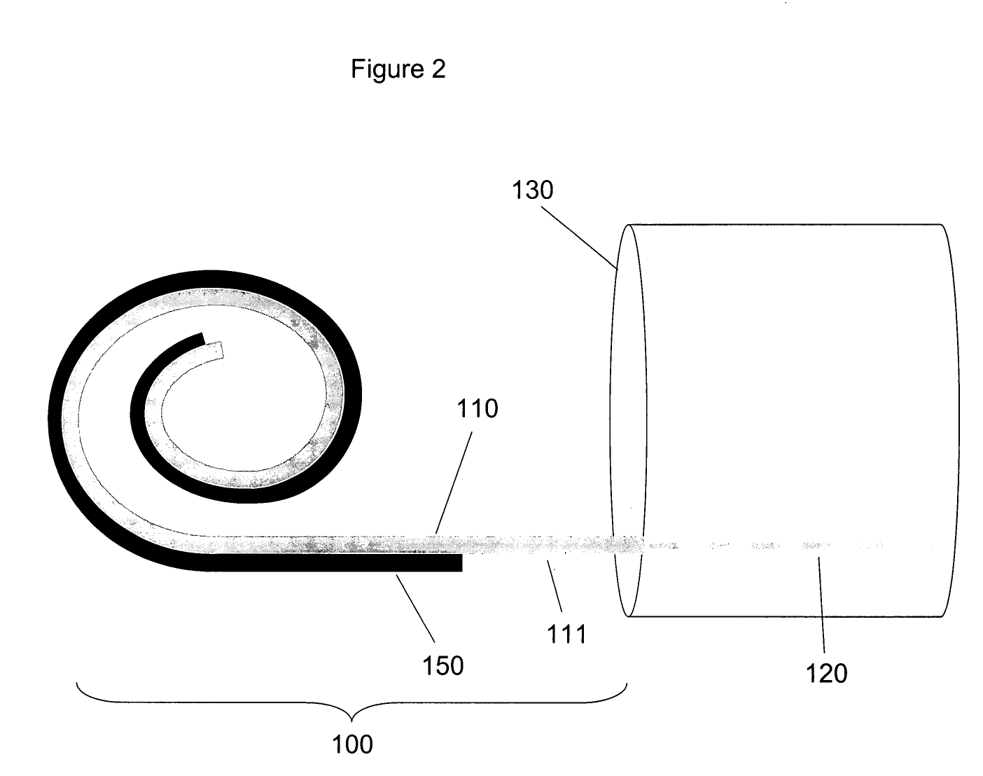

Alternatively, as shown in FIG. 2, delivery mechanism 100 may be used with a sheet, such as a patch or graft, for delivery to tissue. In some embodiments, sheet 150 may be disposed on the outer surface 111 of delivery mechanism 100 for delivery. Sheet 150 is rolled up with delivery mechanism 100 for delivery to a target site. One of ordinary skill in the art would understand that sheet 150 may comprise a patch, graft, implantable plug, or other device. In preferred embodiments, sheet 150 may further comprise a therapeutic agent for delivery of the agent to tissue.

When delivery mechanism 100 is unrolled, a surface of sheet 150 may contact the tissue surface on which it is desired that sheet 150 be delivered. The sheet 150 may be pressed against the desired treatment site by the unrolling mechanism used to unroll delivery mechanism 100, for example due to fluid pressure or other mechanisms. The sheet 150 may be attached to the tissue. One of ordinary skill in the art would understand that there are a variety of means to attach the sheet to the tissue. For example, the sheet 150 may have an adhesive on the patch surface that contacts the tissue. In another embodiment, sheet 150 may be made from shape-memory material, such as Nitinol. When delivery mechanism 100 is unrolled and unconstrained, the memory material may allow sheet 150 to assume the desired shape. Shape-memory material allows an object to return to its initial shape by exposure to external conditions after being deformed to a different shape. For example, a shape-memory material may return to its initial shape when exposed to a minimum temperature. Such a configuration may allow sheet 150 to be given a form comprised of shape-memory material in order to fit sheet 150 to a specific treatment area. Similarly, delivery mechanism 100 may be made from shape memory material such that the material properties of the delivery mechanism will allow it to unroll. For example, in addition to the mechanisms described above to unroll the rolled delivery mechanism, the shape-memory material properties of the rolled delivery mechanism may also be used to unroll the delivery mechanism. One of ordinary skill in the art would understand, for example, that a hot fluid may be injected into a rolled delivery mechanism made from shape-memory material to unroll the delivery mechanism.

In another alternative embodiment, the sheet 150 to be delivered to tissue may be a patch or graft that is folded when rolled within the rolled delivery mechanism, such that when the delivery mechanism 100 is unrolled the patch may unroll and further unfold in order to be placed in a confined region, thereby permitting a patch having a large width to be delivered to the target site. The sheet 150 would be folded onto itself and then rolled up within the delivery mechanism 100 as the catheter 130 is advanced to the target area for delivery of the patch to the diseased muscle. Sheet 150 should be flexible enough such that the patch may stored in its folded position within the rolled delivery mechanism and catheter for delivery.

A person skilled in the pertinent art would also appreciate that the sheet or patch material may include any biostable biocompatible patch material, e.g., polypropylene meshes, metal alloy meshes, titanium metal alloy meshes, and solid metal or polymer disks of material. A patch can also be constructed of materials that have traditionally been used to patch septal defects and aneurysms of the heart, e.g. bovine or equine aldehyde fixed pericardium, polyester and polytetrafluorethylene fabrics, or expanded polytetrafluorethylene (ePTFE). Solid disks of material, e.g. a nonporous disk of plastic or polymer, may allow for attachment of the patch to the muscle surface through suturing or stapling. Nonporous solid disks can have holes used for attaching the patch. Porous disks may allow attachment of the patch with tissue adhesives.

In an alternate embodiment, the outer surface 111 of delivery mechanism 100, when unrolled, may be roughly flat or it may be rounded. The outer surface 111 may be slightly rounded in order to facilitate separation of a patch, graft, or other sheet from the outer surface 111 of the delivery mechanism at the intended treatment site. When delivery mechanism 100 is unrolled, sheet 150 may therefore be more easily removed from delivery mechanism 100. As delivery mechanism 100 is unrolled, the outer longitudinal edge of outer surface 111 may deform from substantially flat to rounded as it unrolls. This deformation may also be accomplished by other means, for example increasing fluid pressure after delivery mechanism 100 has unrolled. As delivery mechanism 100 is deformed, sheet 150 may peel off outer surface 111 and become secured to the intended delivery site.

One or more of the surfaces of sheet 150 and rolled delivery mechanism 100 may be coated with a non-adhesive material, such as Teflon®, in order to lessen bonding between the sheet 150 and the delivery mechanism 100 when rolled together, and will facilitate removal of sheet 150 from the delivery mechanism 100. To facilitate the securing of sheet 150 to a desired treatment location, the outer surface of the sheet 150 that contacts the tissue may be coated with an adhesive. Thus, in an alternate embodiment, the inner surface 110 may be coated with a non-adhesive material to prevent the outer surface of sheet 150 from adhering to the inner surface 110 when rolled together. Additionally or alternatively, outer surface 111 may also be coated with a non-adhesive material to facilitate separation of the sheet 150 from the delivery mechanism 100 once sheet 150 has been positioned and deployed at a treatment location.

A sheet 150 may further comprise a means of securing the sheet 150 to the delivery location. For example, it may comprise an adhesive applied to the outer surface of sheet 150 to adjoin the patch onto tissue. The means to secure sheet 150 may comprise a stake, barb, or other structure mounted on the outer surface of sheet 150. Such devices are described in U.S. patent application Ser. No. 10/121,618, the disclosure of which is incorporated herein by reference.

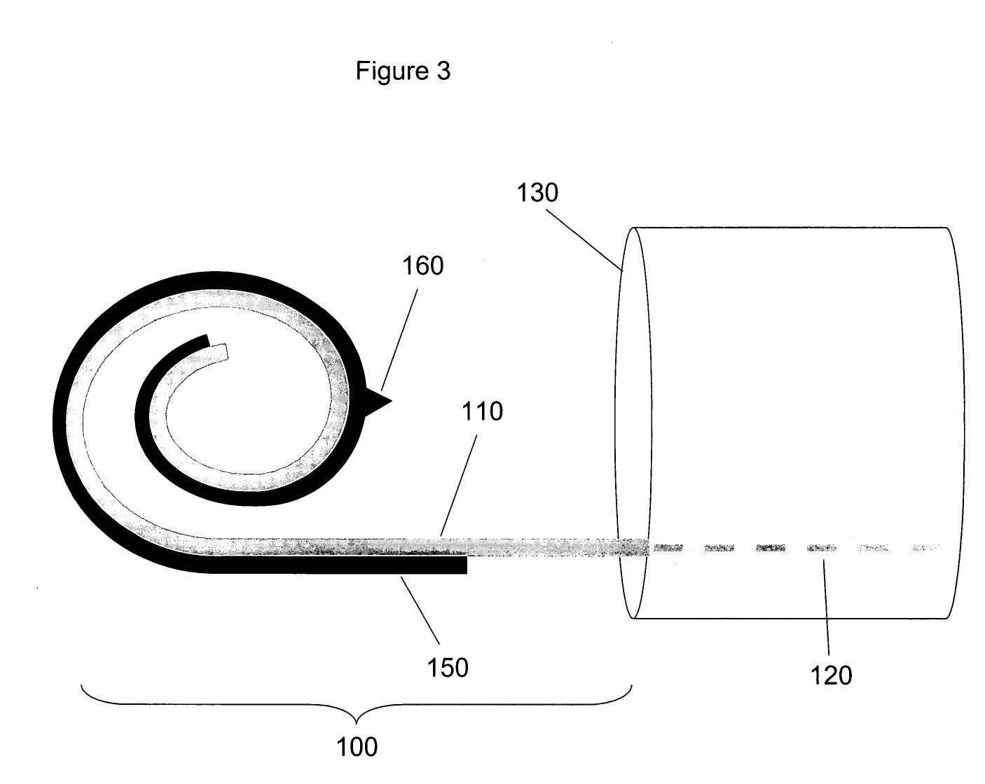

FIG. 3 shows such an alternative embodiment of the present invention. Sheet 150 further comprises stud 160 for positioning and securing sheet 150 to the intended delivery site. The sheet may be a patch, graft, therapeutic agent, or other device. When delivery mechanism 100 is unrolled, stud 160 may contact the region of the intended delivery area. Stud 160 may then attach to or into surrounding tissue in order to secure sheet 150 to the area. When delivery mechanism 100 is withdrawn, sheet 150 may remain at the intended delivery site, for example to deliver time-released therapeutic agent. Stud 160 may secure sheet 150 to the delivery site for a sufficient time for a therapeutic agent to be delivered.

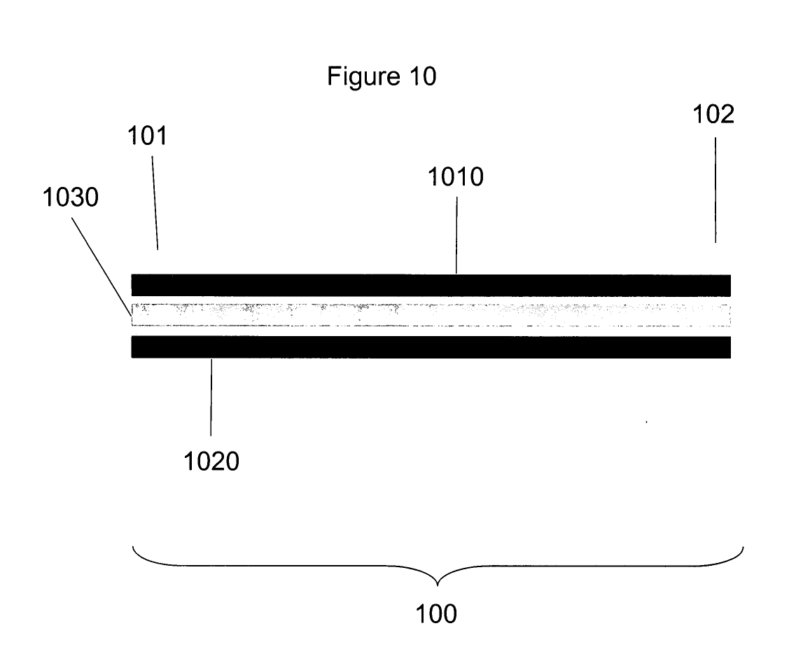

In another embodiment, delivery mechanism 100 may comprise multiple layers. For example, it may comprise two layers of shape-memory material or other material with a third layer disposed between them. The third layer may comprise, for example, a polymer, therapeutic agent, patch, graft, or other substance or device. Multiple layers may be preferred in order to adjust the flexibility, thickness, or other properties of delivery mechanism 100. In some embodiments, the first and second layers may be separable at the distal end 101 (see FIG. 1). When delivery mechanism 100 is unrolled, the third layer may be pushed out of delivery mechanism 100 at an orifice at the distal end 101 in order to be deposited at the intended treatment location. For example, a push rod or wire may be used to first unroll delivery mechanism 100, and then to push the third layer out of unrolled delivery mechanism 100. FIG. 10 shows an enlarged view of such a delivery mechanism 100 after being unrolled. First and second layers 1010 and 1020 are separable at the distal end 101. Third layer 1030 may be pushed out of the distal end 101 by, for example, a wire or push rod (not shown) inserted at proximal end 102.

In another embodiment, the rolled delivery device may itself be a patch, graft, or other device to be adjoined to tissue. The integrated rolled delivery device/patch may be detached from the catheter once it has been unrolled and placed in the desired location. Thus, delivery mechanism 100 of FIG. 1 may itself comprise a patch or graft. When such an integrated delivery mechanism/patch is unrolled, it may contact the intended delivery site. The integrated delivery mechanism/patch may further comprise a means for securing to the delivery site, such as an adhesive, barb, or spike. After the integrated delivery mechanism/patch is unrolled, it may be detached from a catheter (such as catheter 130 shown in FIG. 1) to remain at the desired treatment location. In some embodiments, the entirety of integrated delivery mechanism/patch may detach from catheter. Such configurations may be desirable, for example, to reduce device complexity or cost.

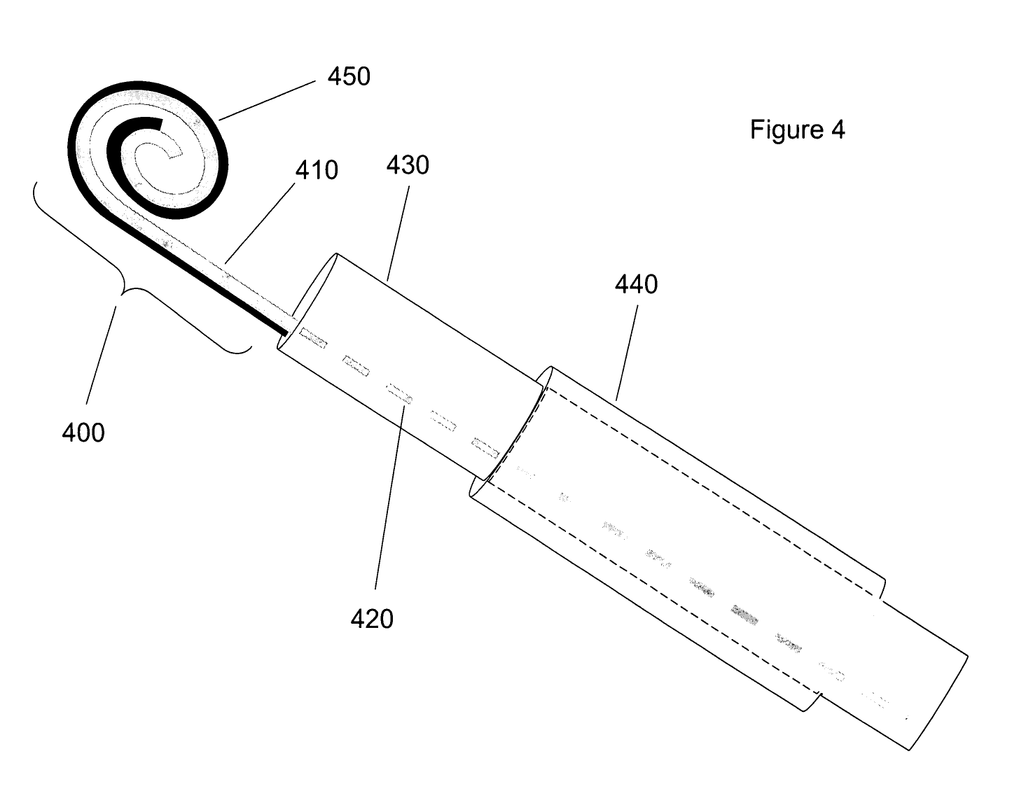

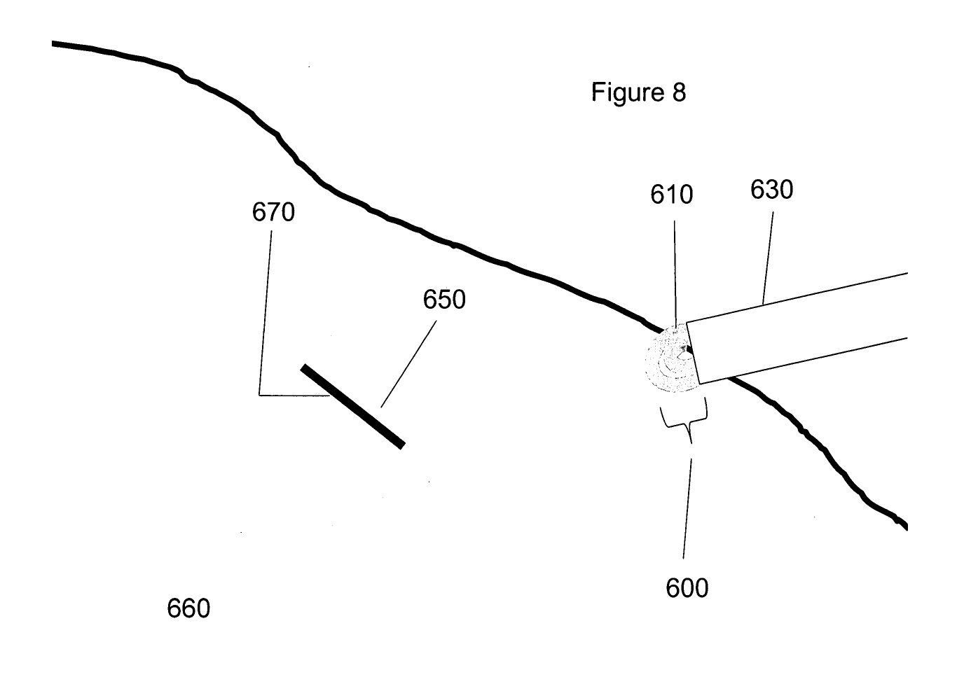

FIG. 4 shows another embodiment of the present invention. A sheath 440 may be concentrically positioned around catheter 430. Delivery mechanism 400 may be disposed at the insertion end of catheter 430, or it may be extendable from within catheter 430 as described previously. Delivery mechanism 400 may comprise rolled delivery mechanism with inner surface 410 and patch 450. Delivery mechanism 400 may be extended as described previously, for example by extending push rod 420. Sheath 440 may be movable along the length of catheter 430, such that sheath 440 may extend past the distal end of catheter 430 to cover delivery mechanism 400 (a delivery mechanism partially covered by a sheath is illustrated in FIG. 8). Sheath 440 may be moved toward the distal end of catheter 430 in order to cover delivery mechanism 400 during insertion of the catheter into a patient. Such a configuration may be desirable in order to provide protection to delivery mechanism 400 or to aid in inserting and positioning catheter 430. Sheath 440 may be moved toward the proximal end of catheter 430 in order to expose delivery mechanism 400 prior to deployment.

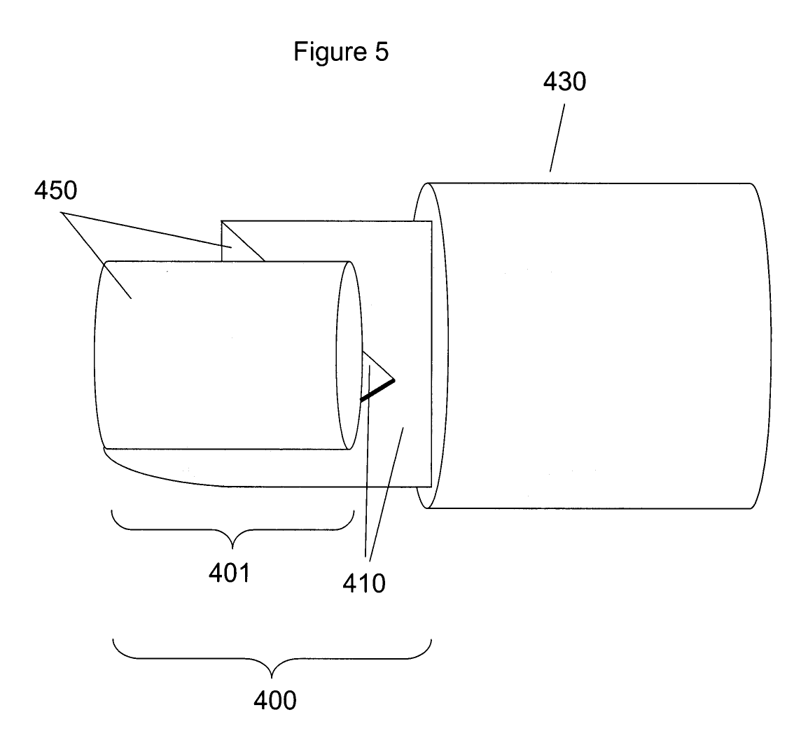

As shown in FIG. 5, in some embodiments of the present invention, delivery mechanism 400 may be rotated 90 degrees in a plane parallel to the plane defined by delivery mechanism 400 when unrolled, prior to being covered by sheath 440. FIG. 5 shows a perspective view of one such embodiment. Rolled portion 401 of delivery mechanism 400 is rotated so that the axis around which it is rolled is parallel to the longitudinal axis of catheter 430. Sheath 440 (not shown) can then be moved over rolled delivery mechanism 400 to constrain and protect it. Such configurations may be preferable in order to reduce the effective diameter of delivery mechanism 400, thereby reducing its profile and facilitating delivery of the rolled delivery device to a target site, and to assist in deploying a sheath over delivery mechanism 400.

When delivery mechanism 400 is unrolled, rolled portion 401 may rotate back to its original position so that the axis around which it is rolled is perpendicular to the longitudinal axis of catheter 430. Delivery mechanism 400 may then be unrolled as previously described. The rotation of rolled portion 401 may be accomplished using the same mechanism used to deploy delivery mechanism 400, such as fluid pressure, mechanical means, or other means (such as shape-memory material characteristics).

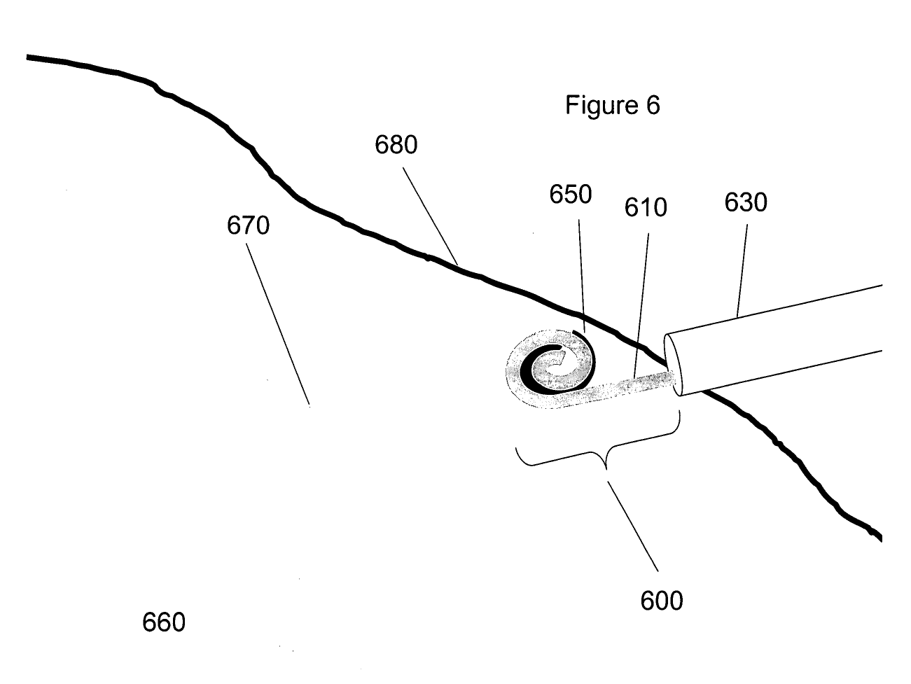

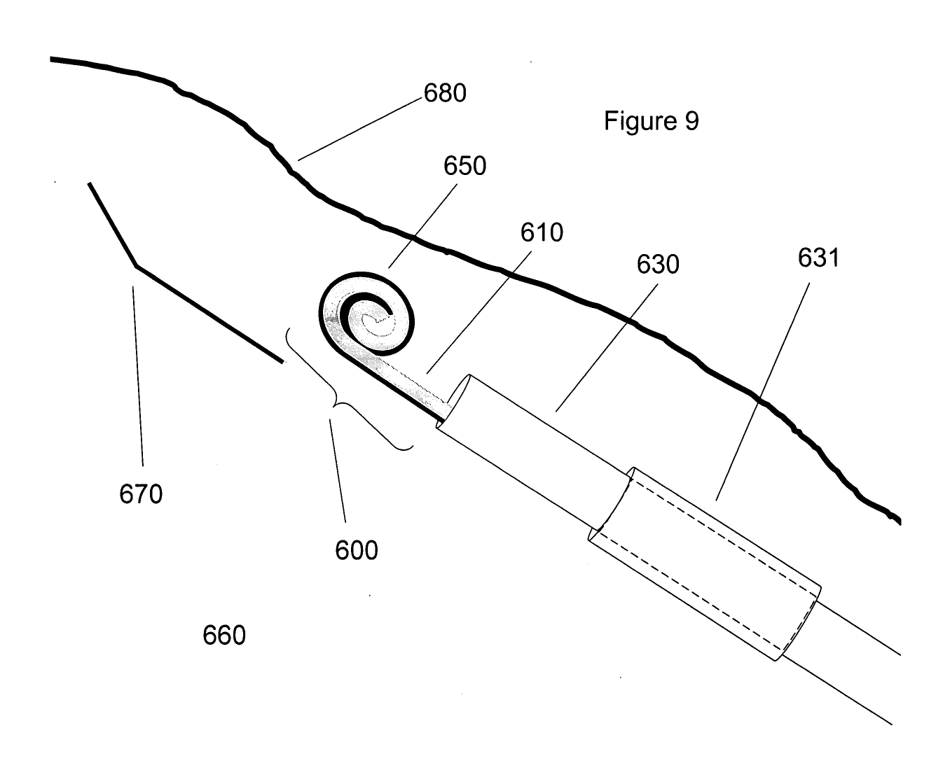

In the operation of the system, illustrated in FIGS. 6 through 8, catheter 630 may be inserted in the region of the area where application of the therapeutic agent is desired 670. Insertion may be made, for example, via arterial or femoral routes, through small openings between the ribs, or through other routes. For example, catheter 630 may be introduced interventionally to a treatment site at the endocardial surface of the heart (i.e., the endocardium or interior muscle of the heart), as shown in FIG. 9, or catheter 630 may be introduced surgically or thorascopically (i.e., through a thoracotomy procedure or open heart surgery) to a treatment site at the epicardial surface of the heart (i.e., the epicardium or exterior muscle of the heart), as shown in FIG. 6. One skilled in the art would appreciate that a thorascope or endoscope may be used instead of a catheter for non-interventional surgical procedures.

Referring to FIG. 6, catheter 630 is positioned near treatment site 670 to deliver a patch, graft, or other device containing therapeutic agent to the selected tissue. Catheter 630 may comprise delivery mechanism 600, which may be attached to the distal end of catheter 630. Catheter 630 may be inserted between, for example, an organ and the surrounding membrane. As a specific example, in FIG. 6 delivery area 660 may represent the myocardium and membrane 680 may represent the pericardium. As will be understood by one skilled in the art, FIGS. 6-8 are shown for illustration purposes and may not be to scale. Once catheter 630 has been placed in or near the desired treatment region, delivery mechanism 600 may be deployed. As shown in FIG. 6, the delivery mechanism 600 and patch 650 are in a rolled configuration. The patch 650 is shown as an example. A patch, graft, sheet, or other device may be used.

Referring to FIG. 7, delivery mechanism 600 may be unrolled using any of the mechanisms previously described. Delivery mechanism 600 may unroll from proximal end 602 such that distal end 601 moves toward the desired treatment site 670. As delivery mechanism 600 is unrolled, fluid pressure, mechanical pressure from, for example, a stiff wire, or other methods of unrolling delivery mechanism 600 may cause patch 650 to be placed at the desired treatment site 670. Patch 650 may adhere to the desired treatment site 670 by using any of the means for attaching the patch to tissue previously described. For example, patch 650 may further comprise an adhesive, stud, or other means to secure patch 650 to desired treatment site 670. Patch 650 may be flexible, to allow the patch to assume a form contoured to desired treatment site 670.

Referring to FIG. 8, after a patch, graft, or other device 650 has been deployed at the desired treatment site 670, delivery mechanism 600 may be withdrawn to the distal end of, or into, catheter 630. Such withdrawal may be accomplished by decreasing fluid pressure within delivery mechanism 600, such that the delivery mechanism is no longer inflated by the fluid pressure. Alternatively, delivery mechanism 600 may comprise a shape-memory material that has an initial rolled shape, such that it returns to the rolled shape when interior fluid pressure is decreased. Delivery mechanism 600 may be withdrawn for example by applying a vacuum through catheter 630, or by means of a stiff wire, pushrod, actuator, or other mechanical means. After delivery mechanism 600 has been withdrawn, catheter 630 may be retracted from the region. Patch 650 may remain attached to intended treatment site 670 in order to deliver time-released therapeutic agent.

As will be understood by one having skill in the art, the above-referenced drawings are for illustration purposes may not be to scale. For example, delivery mechanisms 100, 400, and 600 may be relatively thicker or thinner than shown. They may extend further from the end of the catheter than shown, or not as far as shown. Similarly, other dimensions may be modified from those shown without changing the nature or uses of the device relative to the present invention.

The term “therapeutic agent” as used throughout includes one or more “therapeutic drugs” or “genetic material.” The term “therapeutic agent” used herein includes pharmaceutically active compounds, nucleic acids with and without carrier vectors such as lipids, compacting agents (such as histones), virus (such as adenovirus, adenoassociated virus, retrovirus, lentivirus and a-virus), polymers, hyaluronic acid, proteins, cells and the like, with or without targeting sequences. The therapeutics administered in accordance with the invention includes the therapeutic agent(s) and solutions thereof.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D—Phe—Pro—Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; bAR kinase (bARKct) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factor ÿ and ÿ, platelet-derived endothelial growth factor, platelet-derived growth factor, tumor necrosis factor ÿ, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin-) cells including Lin⁻ CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers, styrene-isobutylene block copolymers such as styrene-isobutylene-styrene tri-block copolymers (SIBS) and other block copolymers such as styrene-ethylene/butylene-styrene (SEBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent/therapeutic agent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

One of skill in the art will realize that the examples described and illustrated herein are merely illustrative, as numerous other embodiments may be implemented without departing from the spirit and scope of the present invention.

What is claimed is:

1. A device for delivering material to tissue comprising:
a rolled delivery mechanism having an inner rolled surface and an outer rolled delivery surface, a proximal end and a distal end, and a longitudinal axis;
a substantially flat delivery sheet having an inner surface and an outer surface, wherein the inner surface is positioned adjacent to the outer rolled delivery surface of the delivery mechanism, and the delivery mechanism and delivery sheet are rolled together along the longitudinal axis from the distal end towards the proximal end to facilitate delivery of the sheet to tissue; and
an unrolling mechanism for unrolling the rolled delivery mechanism, wherein the sheet is delivered to tissue when unrolled.

2. The device of claim 1, further comprising means for attaching the sheet to tissue.

3. The device of claim 1, wherein the outer rolled delivery surface of the delivery mechanism is substantially flat when unrolled.

4. The device of claim 1, wherein the outer rolled delivery surface of the delivery mechanism has at least one rounded edge to facilitate separation of the sheet from the delivery mechanism when unrolled.

5. The device of claim 1, wherein the sheet is a therapeutic patch.

6. The device of claim 1, wherein the sheet is a graft.

7. The device of claim 1, wherein the sheet comprises an implantable plug.

8. The device of claim 1, wherein the sheet carries a therapeutic agent.

9. The device of claim 1, wherein the inner sheet surface is coated with a non-adhesive material to facilitate separation of the sheet from the delivery mechanism when unrolled.

10. The device of claim 1, wherein the unrolling mechanism comprises a push rod.

11. The device of claim 1, wherein the unrolling mechanism comprises an inflatable system in fluid connection with the delivery mechanism.

12. The device of claim 2, wherein the means for attaching the sheet to tissue comprises an adhesive applied to the outer sheet surface.

13. The device of claim 2 wherein the means for attaching the sheet to tissue comprises a stake mounted on the outer sheet surface.

14. The device of claim 2 wherein the means for attaching the sheet to tissue comprises a barb mounted on the outer sheet surface.

15. The device of claim 1, further comprising a means for delivering the delivery mechanism to a target site.

16. The device of claim 15, wherein the means for delivering comprises a catheter.

17. The device of claim 15, wherein the means for delivering comprises an endoscope.

18. The device of claim 15, wherein the means for delivering comprises a thorascope.

19. The device of claim 1, wherein the rolled delivery mechanism is made from shape-memory material.

20. The device of claim 19, wherein the means for unrolling the rolled delivery mechanism comprises the application of heat.

21. The device of claim 1, further comprising a sheath, wherein the sheath is concentrically positioned around the delivery mechanism.

22. The device of claim 21, wherein the delivery mechanism and sheet are rolled together and turned in a longitudinal position within the sheath to facilitate delivery of the delivery mechanism.

23. A device for delivering a therapeutic agent to tissue comprising:
a rolled delivery mechanism having an inner rolled surface and an outer rolled delivery surface, a proximal end and a distal end, and a longitudinal axis,
wherein the outer rolled delivery surface is coated with a therapeutic agent for delivery of the therapeutic agent onto tissue, and the delivery mechanism is rolled along the longitudinal axis from the distal end towards the proximal end; and
an unrolling mechanism for unrolling the rolled delivery mechanism, wherein the therapeutic agent is delivered to tissue when unrolled.

24. The device of claim 23, further comprising a means for delivering the delivery mechanism to a target site.

25. The device of claim 24, wherein the means for delivering comprises a catheter.

26. The device of claim 24, wherein the rolled delivery mechanism can detach from the means for delivering the delivery mechanism.

27. The device of claim 26, wherein the rolled delivery mechanism is a patch.

28. The device of claim 26, wherein the rolled delivery mechanism further comprises a means for attaching the delivery mechanism to tissue.
